(19) United States
(12) Patent Application Publication
(10) Pub. No.: US 2003/0225326 A1
(43) Pub. Date: Dec. 4, 2003

(54) Methods and combinations relating to information obtained using brain imaging techniques

(76) Inventors: Bernard Querleux, Le Perreux (FR); Yves Burnod, L'Hay-Les-Roses (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/405,646

(22) Filed: Apr. 3, 2003

Related U.S. Application Data
(60) Provisional application No. 60/383,626, filed on May 29, 2002.

(30) Foreign Application Priority Data
Apr. 3, 2002 (FR) 02 04142

(51) Int. Cl. A61B 5/05

(52) U.S. Cl. 600/407; 600/410; 600/409; 600/544; 600/476; 600/557

(57) ABSTRACT

Methods and combinations relate to information obtained by using a brain imaging technique. In one example, there is a method of determining information reflecting impact of a product on skin sensitivity. Such a method includes causing at least one stimulus to be applied to skin of a subject, the stimulus being capable of provoking a brain reaction reflecting the presence of sensitive skin; receiving first information obtained using a brain imaging technique, the first information relating to the reaction of at least one brain region to the stimulus; causing a product to be applied or administered to the subject, the product being a product intended to impact skin sensitivity; receiving second information obtained using a brain imaging technique, the second information relating to behavior of the brain region after application or administration of the product to the subject; and determining third information as a function of the first and second information, the third information relating to the impact of the product on skin sensitivity.

This application claims the benefit of priority under 35 U.S.C. §119(e) of U.S. Provisional Patent Application No. 60/383,626, filed May 29, 2002.

The present invention pertains to methods and combinations relating to information obtained using one or more brain imaging techniques. Among other things, the present invention relates to treating sensitive skin, evaluating skin sensitivity, and/or associating one or more products, such as cosmetics, with information obtained via at least one brain imaging technique.

### BACKGROUND

Most women consider their skin to be sensitive. Nevertheless, in the view of at least some dermatologists, this opinion is sometimes more associated with considerations of a psychological type than with any physiological reality. In other words, many woman may think their skin is sensitive but there may not exist actual physical basis for that belief.

This makes it difficult to evaluate the effectiveness of products and treatments for various types of sensitive skin. This also reveals the need for one or more methods of evaluating skin sensitivity that can be used for validating, in an objective manner, the assessment made by an individual.

### SUMMARY

Although the present invention may obviate one or more needs or the related art discussed above, it should be understood that the invention might not necessarily obviate one or more of those needs.

In the following description, certain aspects and embodiments will become evident. It should be understood that the invention, in its broadest sense, could be practiced without having one or more features of these aspects and embodiments. It should also be understood that these aspects and embodiments are merely exemplary.

In one aspect, as embodied and broadly described herein, the invention may include a method of determining information reflecting impact (e.g., effectiveness) of a product on skin sensitivity. The method may include causing at least one stimulus to be applied to skin of a subject, the stimulus being capable of provoking a brain reaction reflecting the presence of sensitive skin. The method may also include receiving first information obtained using a brain imaging technique, the first information relating to the reaction of at least one brain region to the stimulus. A product may be caused to be applied or administered to the subject, the product being a product intended to impact skin sensitivity. The method may also include receiving second information obtained using a brain imaging technique, the second information relating to the behavior of the brain region after application or administration of the product to the subject. The method may further include determining third information as a function of the first and second information, the third information relating to the impact of the product on skin sensitivity.

In some examples, the method may include outputting the third information.

For some examples, the determining may include comparing the first and second information.

In another aspect, the method may include causing the skin to be stimulated again, wherein the second information relates to the behavior of the brain region after the skin is stimulated again. In some examples, this may reveal different behaviors of the brain in response to a given stimulus, in the presence or the absence of action from the product. The action of the product may be considered as being demonstrated, for example, when the activity of the brain in response to a discomfort-generating stimulus is smaller after the product has been applied or administered than it was before. For example, the product may have an effect on how the stimulus is transmitted to the brain, e.g. by reducing the sensitivity of the nerve terminations in the skin.

The product may be applied or administered after the first information is obtained using the brain imaging technique. If the action of the product is slow to come into effect, it may also be applied or administered immediately prior to obtaining the first information, i.e. before the product has had sufficient time to produce its effect(s).

In another aspect, the brain imaging technique used to obtain the second information may be capable of revealing whether or not the reaction of the brain region to the stimulus is reduced after the product has been applied or administered.

In yet another aspect, there may be a method of marketing a product, including performing any one of the methods described herein and providing a notification indicating that the product has been tested by using a brain imaging technique.

In one other aspect, there is a method of evaluating the sensitivity of skin. The method may include receiving first information obtained using a technique other than a brain imaging technique, the first information relating to the sensitivity of skin of at least one subject. The method may also include causing at least one stimulus to be applied to skin of the at least one subject, and receiving second information obtained using a brain imaging technique, the second information relating to the reaction of at least one brain region to the stimulus. The method may further include determining third information as a function of the first and second information.

In still another aspect, the determining may include determining the third information as a function of the degree of correlation between the first and second information.

In a further aspect, the third information may include information confirming or not confirming the first information.

In an even further aspect, the first information may be obtained by means of a questionnaire.

One more aspect relates to a method of selecting subjects for testing. Such a method may involve performing any of the methods disclosed herein for a plurality of subjects, and selecting at least some of the subjects based on the third information.

In yet another aspect, the method may include subjecting said at least some subjects to testing, wherein the testing may include testing used to devise and/or evaluate at least one cosmetic.

One further aspect relates to a method of marketing a product, wherein the method may include testing a product by using at least one subject found to have sensitive skin by performing any of the methods discussed herein. The testing may comprise applying the product to skin, and the method may further include providing a notification indicating that the product has been tested using at least one subject found to have sensitive skin.

An even further aspect relates to a method of assisting in formulation and/or selection of a cosmetic. Such a method may include establishing a relationship between a cosmetic and a stimulus for a subject; receiving information obtained using a brain imaging technique, the information relating to the reaction of at least one brain region to the stimulus; and performing at least one of the following: determining at least one modification to the formulation of a cosmetic as a function of the information, and selecting at least one product from among a plurality of products as a function of the information.

In some examples of the above-mentioned method, it may be possible to formulate or to select a cosmetic in such a manner that while it is being applied a brain reaction is obtained that corresponds as closely as possible to the intended purpose, for example a feeling of well-being or calm.

According to another aspect, the stimulus may be a predetermined stimulus, and/or the at least one brain region may be at least one predetermined brain region

In one more aspect, the causing of at least one stimulus to be applied to skin of a subject, may include causing an irritating substance to be applied to the skin. Examples of irritating substance include substances comprising at least one of lactic acid and capsaicin. In some example, the stimulus could be a stimulus other than a stimulus in the form of an irritating substance.

As used herein, “causing” an action (e.g., causing at least one stimulus to be applied to skin) may refer to one or more of a direct act of performing the action, and any indirect act of facilitating, encouraging, or being an accessory to the action. Thus, causing at least one stimulus to be applied to skin may include applying the stimulus to the skin. It may also (or alternately) include providing instruction(s) and/or direction(s) for performing the action, and/or providing some form of means (e.g., an irritating substance and/or an implement) by which the stimulus may be applied.

In yet another aspect, the at least one region of the brain may include the primary sensory contralateral cortex, the superior prefrontal cortex, and/or the cingulate region.

Any type of brain imaging technique may be used to obtain information. For example, the brain imaging technique may be a functional magnetic resonance imaging technique (fMRI). Other possible technique include a positron-emission camera imaging technique, an electroencephalographic (EEG) imaging technique, an netoencephalographic (MEG) imaging technique, an optical imaging technique, or any other imaging technique. Examples of using a brain imaging technique are discussed in the following: “Brain processing of capsaicin-induced secondary hyperalgesia: a functional MRI study,” published in Neurology, Aug. 11, 1999; 53(3): 548-57, and “Functional reorganization of the human primary somatosensory cortex after acute pain demonstrated by magnetoencephalography,” published in Neuroscience Letters, Feb. 9, 2001, 298(3): 195-8.

A further aspect relates to a method of marketing and/or promoting sales of a product (e.g., cosmetic), wherein the method includes providing a notification indicating that the product has been tested by using a brain imaging technique.

An additional aspect relates to a combination including a product (e.g., a cosmetic) and a notification indicating that the product has been tested by using a brain imaging technique.

The product may be a product for application to the skin, e.g. a cream, a lotion, an ointment, an oil, a powder, or a product in any other form. For example, the product may be contained in a medium for application to the skin, e.g., a patch, a dressing, a bandage, a mask, or any other medium.

In some examples, the product may be a cosmetic. One exemplary definition of a cosmetic is contained in Counsel Directive 93/35/EEC (European Economic Community) dated Jun. 14, 1993. At least some cosmetics could be used to perform a cosmetic treatment.

In some examples, the product may be product intended to impact skin sensitivity. Some exemplary products may be intended primarily to reduce skin sensitivity. Other exemplary products may not be intended solely for reducing skin sensitivity. By way of example, the product may be intended mainly to provide makeup, hydration, skin protection (e.g., protecting the skin against sunlight and/or depigmenting), and/or skin repair, and it may also have effects on skin sensitivity. Such products may contain various compounds (e.g., active agents) other than compounds for reducing skin sensitivity.

Among the active agents that may be used for reducing skin sensitivity, those which act on nerve terminations may be suitable for use. Active agents intended to produce a well being, softening, and/or calming sensation may be used.

One example of a product having an action on skin sensitivity includes substance P antagonists and/or extracts from at least one plant of the Rosaceae family, as described in European patent application No. EP 0 906 752.

In some examples, the product may be a non-cosmetic. For example, the product may comprise an item, object, tangible merchandise, good, and/or any other form of product that might possibly lead to brain activity detectable via a brain imaging technique. Further, the product may be a product that is not applied to skin. For example, the product may be applied to hair, nails, or any other portion of the body. Alternatively, the product may evoke detectable brain activity via taste, smell, touch, sight, or other interaction with the product and the evoked brain activity may reveal information regarding the subject's interaction with that product.

The information relating to the behavior of the brain may be received in various ways (e.g., visually, audibly, electronically, and/or via any form of communication means, such as those discussed below in connection with the notification). For example, the information may include raw data and/or data resulting from prior processing, e.g. for quantifying the intensity and/or the extent and/or the rate of activity of one or more brain areas. The information may comprise an image and/or data that may be processed to construct an image.

The notification could be in any form and could be performed using any means of transferring (e.g., communicating) information. For example, the notification may include printed material associated with the product. The printed material may include at least one of printed material appearing on packaging associated with (e.g., containing) the product, printed material (e.g., instructions) contained in packaging associated with (e.g. containing) the product, a product label, a display at a point of sale for the product, an advertisement, a sign, a mailing, and a flyer. The notification may be any form of written press or any other document, such as those for advertising purposes.

The notification may include a broadcasted advertisement. Examples of broadcast advertisements include an electronic mail (email), a website page, a web cast, an audio broadcast, an analog video broadcast, a digital video broadcast, a television broadcast, a radio broadcast, a broadcast via a telephone network (e.g., a mobile phone network), a broadcast via a computer network, a broadcast via the Internet, and any other form of broadcast. The notification may be conveyed by a sales person directly at a point of sale, by radio, by TV, or by telephone. The notification may be in the form of advertising spots or short messages.

In another aspect, the notification may indicate that the effectiveness of the product has been verified by using a brain imaging technique.

In a further aspect, the notification may include a notification at a point of sale for the product.

Aside from the structural and procedural arrangements set forth above, the invention could include a number of other arrangements, such as those explained hereinafter. It is to be understood, that both the foregoing description and the following description are exemplary.

### BRIEF DESCRIPTION OF DRAWINGS

The accompanying drawings are incorporated in and constitute a part of this specification. The drawings illustrate exemplary embodiments of the invention and, together with the description, serve to explain some principles of the invention. In the drawings,

FIG. 1 shows a partially schematic, perspective view of an exemplary apparatus that could be used to obtain information in a brain imaging technique for use in a method and/or combination in accordance with embodiments of the invention;

FIG. 2 shows an exemplary view of a brain image, obtained using the apparatus of FIG. 1, for a subject considered not to have sensitive skin;

FIG. 3 shows an exemplary view of a brain image, obtained using the apparatus of FIG. 1, for a subject considered to have sensitive skin;

FIG. 4 shows a exemplary graph plotting discomfort values over time for different groups of subjects; and

FIG. 5. schematically shows an exemplary combination of a product and a notification indicating that the product has been tested using a brain imaging technique.

### DESCRIPTION OF EXEMPLARY EMBODIMENTS

Reference will now be made in detail to some possible embodiments of the invention, examples of which are illustrated in the accompanying drawings. Wherever possible, the same reference numbers are used in the drawings and the description to refer to the same or like parts.

In one exemplary method incorporating certain features of the invention, a test is performed by submitting a questionnaire to a group of individuals, wherein the questionnaire includes questions suitable for evaluating skin sensitivities of the people in the group.

The questionnaire may include a variety of questions. Such questions may be intended to elicit responses indicative of whether climatic or topical factors generate discomfort. For example, the questions may inquire about whether an individual's skin is sensitive to various factors, such as cold, sudden changes in temperature, and/or hard water. The questionnaire may possibly also include questions relating to discomfort caused by coming into contact with certain materials or application of certain products.

The replies to the questionnaire may be processed to select at least two subgroups of people, a first subgroup made up of people who can be considered as having sensitive skin and a second subgroup of people who can be considered as not having sensitive skin. Each subgroup comprises, for example, nine people.

The exemplary method could include acquiring initial anatomical brain images for each of the people in the subgroups. Thereafter, an irritating product (e.g., a product containing a substance that generates discomfort, such as, e.g., lactic acid or capsaicin mixed with a vehicle, e.g., a saline solution) may be applied to a set of people from each subgroup, and the brain reactions of those people may be observed by a brain imaging technique, such as, for example, functional nuclear magnetic resonance imaging (fMRI). Control tests may also be performed by applying an inert substance, which may be a vehicle substance (e.g., saline) alone. For each of at least some of the people in the subgroups, the irritating product may be applied to one skin portion (e.g., a portion of the face, such as one-half of the face) while the inert substance may be applied to another skin portion. For purposes of facilitating an explanation of the following example, it may be assumed that the irritating product is applied to the right side of the nose and the vehicle alone is applied to the left side of the nose.

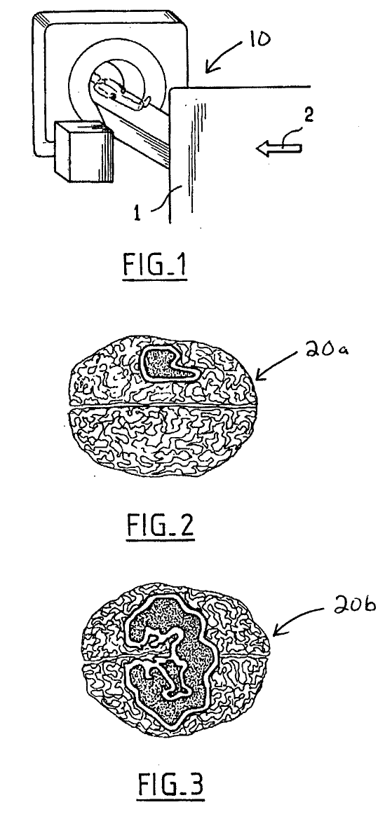

While acquiring brain images by fMRI, each person may lie in a prone position in an apparatus 10 as shown diagrammatically in FIG. 1. The apparatus 10 may be equipped with fMRI imaging capabilities and include a mirror enabling a person being imaged to observe a screen 1 on which an arrow 2 is projected. The arrow 2 may be controlled to point in either the left direction or the right direction, for example.

The apparatus 10 may also include an input device (not shown), such as a keypad or any other input device, for enabling a subject being imaged to indicate whether that person feels discomfort on the left side or the right side of the nose, depending on the direction in which the arrow 2 is pointing. In addition, such an input device may be configured to enable an individual to input the relative degree of any discomfort. In one example, for a period of 10 minutes, images of brain activity may be obtained once every 3 seconds, and at an interval of once every 16 seconds, the direction of the displayed arrow 2 may be changed in a random manner.

Each person may use the input device to input a response in a range of 0 to 3 depending on the level of discomfort that is felt.

FIG. 2 shows an example of a brain image for a person considered as not having sensitive skin, and FIG. 3 shows an example of a brain image for a person considered as having sensitive skin.

The application of the irritating substance may cause brain activity to appear on the primary contralateral cortex for the people of the first subgroup (i.e., those who can be considered as having sensitive skin) while no similar activity is observed during control tests. Extended brain activity located, for example, in the prefrontal superior cortex and in the cingulate region, may be observed for the people of the first subgroup, whereas the activity may smaller among the people of the second subgroup (i.e., those who can be considered as not having sensitive skin), thus revealing correlation between the replies to the questionnaire and physiological reality.

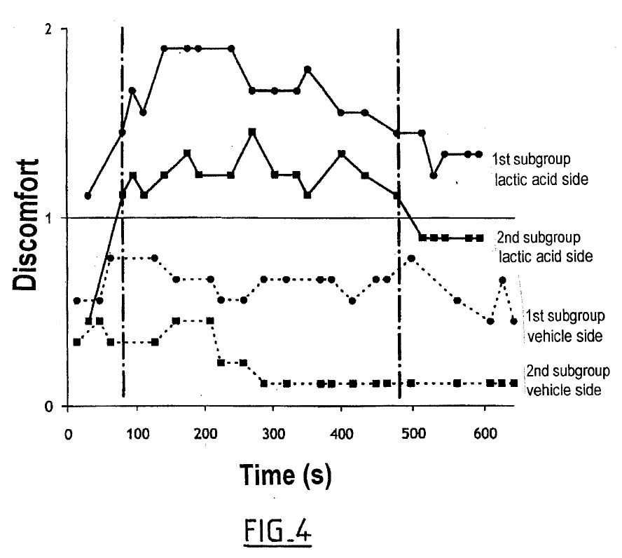

FIG. 4 plots discomfort values as a function of time for people in the first and second subgroups, depending on whether the arrow 2 of FIG. 1 is pointing to the side on which irritating substance (e.g., lactic acid) was applied or to the side on which the vehicle alone was applied.

It can be seen that for people having sensitive skin (i.e., people in the first subgroup), the experienced discomfort is greatest between the starting and ending stages of the experiment as defined by vertical chain-dotted lines in FIG. 4.

Methods relating to the above-mentioned examples may be used for a variety of different purposes. For example, such methods may include methods of evaluating skin sensitivity and/or selecting subjects for testing. Other examples of such methods may include methods of determining information reflecting impact of a product on skin sensitivity, and/or formulating and/or selecting a product, wherein such methods may involve brain scan images obtained after a product is applied or administered to a subject.

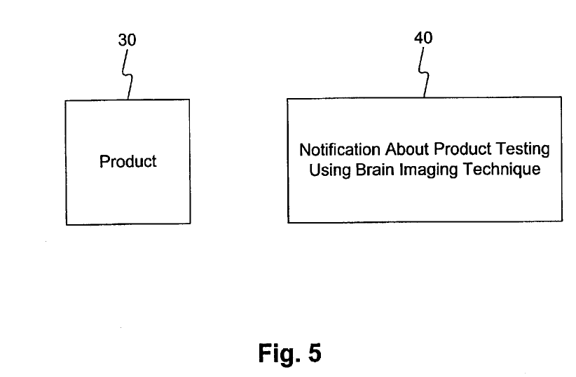

FIG. 5 shows an exemplary embodiment of a combination according to the invention, which includes a product 30 and a notification 40 indicating that the product has been tested using a brain imaging technique. The product 30 may be any type of product capable of being applied or administered to a subject. For example, the product 30 may be a cosmetic product, such as a cosmetic comprises a product intended to impact skin sensitivity.

The notification 40 may be in any form. In one example, the notification 40 may comprise printed material associated with the product. Examples of such printed material include printed material appearing on packaging containing the product, printed material contained in packaging containing the product, a product label, a display, an advertisement, a sign, a mailing, a flyer, and any other form of printed material. The notification 40 may also include a broadcasted advertisement. Examples of broadcasted advertisements include an email, a website page, a web cast, an audio broadcast (including a person to person, live announcement), an analog video broadcast, a digital video broadcast, a television broadcast, a radio broadcast, a broadcast via a telephone network, a broadcast via a computer network, a broadcast via the Internet, and any other form of broadcasted announcement. In some examples, the notification may include a notification at a point of sale for the product

Throughout the description, including in the claims, the expression “a” should be understood as being synonymous with “at least one” (i.e., relating to both the singular and the plural) unless otherwise specified to the contrary.

It will be apparent to those skilled in the art that various modifications and variations can be made to the structure and methodology of the present invention. Thus, it should be understood that the invention is not limited to the examples discussed in the specification. Rather, the present invention is intended to cover modifications and variations.

What is claimed is:

1. A method of determining information reflecting impact of a product on skin sensitivity, comprising:
causing at least one stimulus to be applied to skin of a subject, the stimulus being capable of provoking a brain reaction reflecting the presence of sensitive skin;
receiving first information obtained using a brain imaging technique, the first information relating to the reaction of at least one brain region to the stimulus;
causing a product to be applied or administered to the subject, the product being a product intended to impact skin sensitivity;
receiving second information obtained using a brain imaging technique, the second information relating to behavior of the brain region after application or administration of the product to the subject; and
determining third information as a function of the first and second information, the third information relating to the impact of the product on skin sensitivity.

2. A method according to claim 1, wherein the stimulus is a predetermined stimulus, and wherein the at least one brain region is at least one predetermined brain region.

3. A method according to claim 1, wherein the method further comprises outputting the third information.

4. A method according to claim 1, wherein the determining comprises comparing the first and second information.

5. A method according to claim 1, further comprising causing the skin to be stimulated again, the second information relating to the behavior of the brain region after the skin is stimulated again.

6. A method according to claim 1, wherein the brain imaging technique used to obtain the second information is capable of revealing whether or not the reaction of the brain region to the stimulus is reduced after the product has been applied or administered.

7. A method according to claim 1, wherein causing at least one stimulus to be applied to skin of a subject comprises causing an irritating substance to be applied to the skin.

8. A method according to claim 7, wherein the irritating substance comprises at least one of lactic acid and capsaicin.

9. A method according to claim 1, wherein the at least one region of the brain comprises at least one of the primary sensory contralateral cortex, the superior prefrontal cortex, and the cingulate region.

10. A method according to claim 1, wherein the imaging technique used to obtain at least one of the first and second information comprises a functional magnetic resonance imaging technique.

11. A method according to claim 1, wherein the imaging technique used to obtain at least one of the first and second information comprises at least one of a positron-emission camera imaging technique, an EEG imaging technique, an MEG imaging technique, and an optical imaging technique.

12. A method according to claim 1, wherein the product is a cosmetic.

13. A method of marketing a product, comprising:
performing the method of claim 1; and
providing a notification indicating that the product has been tested by using a brain imaging technique.

14. A method according to claim 13, wherein the notification indicates that the effectiveness of the product has been verified by using a brain imaging technique.

15. A method according to claim 13, wherein the product is a cosmetic.

16. A method according to claim 13, wherein the notification comprises printed material associated with the product.

17. A method according to claim 16, wherein the printed material comprises at least one of printed material appearing on packaging associated with the product, printed material contained in packaging associated with the product, a product label, a display at a point of sale for the product, an advertisement, a sign, a mailing, and a flyer.

18. A method according to claim 13, wherein the notification comprises a broadcasted advertisement.

19. A method according to claim 18, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

20. A method according to claim 13, wherein the notification comprises a notification at a point of sale for the product.

21. A method of evaluating the sensitivity of skin, comprising:
receiving first information obtained using a technique other than a brain imaging technique, the first information relating to the sensitivity of skin of at least one subject;
causing at least one stimulus to be applied to skin of the at least one subject;
receiving second information obtained using a brain imaging technique, the second information relating to the reaction of at least one brain region to the stimulus; and
determining third information as a function of the first and second information.

22. A method according to claim 21, wherein the stimulus is a predetermined stimulus, and wherein the at least one brain region is at least one predetermined brain region.

23. A method according to claim 21, wherein the determining comprises determining the third information as a function of the degree of correlation between the first and second information.

24. A method according to claim 21, wherein the third information comprises information confirming or not confirming the first information.

25. A method according to claim 21, wherein causing at least one stimulus to be applied to skin comprises causing an irritating substance to be applied to the skin.

26. A method according to claim 25, wherein the irritating substance comprises at least one of lactic acid and capsaicin.

27. A method according to claim 21, wherein the at least one region of the brain comprises at least one of the primary sensory contralateral cortex, the superior prefrontal cortex, and the cingulate region.

28. A method according to claim 21, wherein the imaging technique used to obtain the second information comprises a functional magnetic resonance imaging technique.

29. A method according to claim 21, wherein the imaging technique used to obtain the second information comprises at least one of a positron-e mission camera imaging technique, an EEG imaging technique, an MEG imaging technique, and an optical imaging technique.

30. A method according to claim 21, wherein the first information is obtained via a questionnaire.

31. A method of selecting subjects for testing, the method comprising:
performing the method of claim 21 for a plurality of subjects; and
selecting at least some of the subjects based on the third information.

32. A method according to claim 31, further comprising subjecting said at least some subjects to testing, wherein the testing comprises testing used to devise and/or evaluate at least one cosmetic.

33. A method of marketing a product, the method comprising:
testing a product by using at least one subject found to have sensitive skin by performing the method of claim 21, wherein the testing comprises applying the product to skin;
providing a notification indicating that the product has been tested using at least one subject found to have sensitive skin.

34. A method according to claim 33, wherein the product is a cosmetic.

35. A method according to claim 33, wherein the notification comprises printed material associated with the product.

36. A method according to claim 35, wherein the printed material comprises at least one of printed material appearing on packaging associated with the product, printed material contained in packaging associated with the product, a product label, a display at a point of sale for the product, an advertisement, a sign, a mailing, and a flyer.

37. A method according to claim 33, wherein the notification comprises a broadcasted advertisement.

38. A method according to claim 37, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

39. A method according to claim 33, wherein the notification comprises a notification at a point of sale for the product.

40. A method of marketing a cosmetic, comprising:
providing a notification indicating that the cosmetic has been tested by using a brain imaging technique.

41. A method according to claim 40, wherein the notification indicates that the effectiveness of the cosmetic has been verified by using a brain imaging technique.

42. A method according to claim 41, wherein the notification comprises printed material associated with the cosmetic.

43. A method according to claim 42, wherein the printed material comprises at least one of printed material appearing on packaging associated with the cosmetic, printed material contained in packaging associated with the cosmetic, a product label, a display at a point of sale for the cosmetic, an advertisement, a sign, a mailing, and a flyer.

44. A method according to claim 41, wherein the notification comprises a broadcasted advertisement.

45. A method according to claim 44, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

46. A method according to claim 41, wherein the notification comprises a notification at a point of sale for the cosmetic.

47. A method of promoting sales of a product, the method comprising:
providing a notification indicating that the product has been tested by using a brain imaging technique.

48. A method according to claim 47, wherein the notification indicates that the effectiveness of the product has been verified by using a brain imaging technique.

49. A method according to claim 47, wherein the product is a cosmetic.

50. A method according to claim 47, wherein the notification comprises printed material associated with the product.

51. A method according to claim 50, wherein the printed material comprises at least one of printed material appearing on packaging associated with the product, printed material contained in packaging associated with the product, a product label, a display at a point of sale for the product, an advertisement, a sign, a mailing, and a flyer.

52. A method according to claim 47, wherein the notification comprises a broadcasted advertisement.

53. A method according to claim 52, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

54. A method according to claim 47, wherein the notification comprises a notification at a point of sale for the product.

55. A method of assisting in formulation and/or selection of a cosmetic, comprising:
establishing a relationship between a cosmetic and a stimulus for a subject;
receiving information obtained using a brain imaging technique, the information relating to the reaction of at least one brain region to the stimulus; and
performing at least one of the following:
determining at least one modification to the formulation of a cosmetic as a function of the information and
selecting at least one product from among a plurality of products as a function of the information.

56. A combination comprising:
a cosmetic; and
a notification indicating that the cosmetic has been tested by using a brain imaging technique.

57. A combination according to claim 56, wherein the cosmetic comprises a substance intended to impact skin sensitivity.

58. A combination according to claim 56, wherein the notification comprises printed material associated with the product.

59. A combination according to claim 58, wherein the printed material comprises at least one of printed material appearing on packaging associated with the product, printed material contained in packaging associated with the product, a product label, a display at a point of sale for the product, an advertisement, a sign, a mailing, and a flyer.

60. A combination according to claim 56, wherein the notification comprises a broadcasted advertisement.

61. A combination according to claim 60, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

62. A combination according to claim 58, wherein the notification comprises a notification at a point of sale for the product.

63. A combination comprising:
a product; and
a notification indicating that the product has been tested by using a brain imaging technique, wherein the notification comprises a notification at a point of sale for the product.

64. A combination according to claim 63, wherein the product comprises a substance intended to impact skin sensitivity.

65. A combination according to claim 63, wherein the notification comprises printed material associated with the product.

66. A combination according to claim 65, wherein the printed material comprises at least one of printed material appearing on packaging associated with the product, printed material contained in packaging associated with the product, a product label, a display at a point of sale for the product, an advertisement, a sign, a mailing, and a flyer.

67. A combination according to claim 63, wherein the notification comprises a broadcasted advertisement.

68. A combination according to claim 67, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

69. A combination according to claim 63, wherein the notification comprises a notification at a point of sale for the product.
